(19) United States
(12) Patent Application Publication
(10) Pub. No.: US 2022/0175982 A1
(43) Pub. Date: Jun. 9, 2022

(54) METHODS AND DEVICES FOR SANITIZATION

(71) Applicants: Stephen Almeida, Tampa, FL (US); James Kerr, Scarborough, ME (US)

(72) Inventors: Stephen Almeida, Tampa, FL (US); James Kerr, Scarborough, ME (US)

(21) Appl. No.: 17/113,086

(22) Filed: Dec. 6, 2020

(51) Int. Cl. A61L 2/10; A61L 2/24; A23L 5/30

(52) U.S. Cl. CPC A61L 2/10; A61L 2/24; A23L 5/36; A61L 2202/11; A61L 2202/121; A61L 2202/14; A61L 2202/24; A61L 2202/23; A61L 2202/122

(57) ABSTRACT

The present invention relates to sanitization devices and methods. More particularly, the invention relates to sanitizing devices and methods that significantly reduce, deactivate or eliminate germs, bacteria and/or other pathogenic microorganisms from objects that have been placed in sanitizing in the sanitizing devices, which are sanitizing bags and sanitizing containers of the current invention, such devices include large and small containers. The device and method use germicidal electromagnetic radiation.

### FIELD OF INVENTION

The present invention relates to sanitization devices and methods. More particularly, the invention relates to devices and methods that significantly reduce, deactivate or eliminate germs, bacteria and/or other pathogenic microorganisms from objects that are contained in the sanitation devices, such devices include large and small containers. The device and method use germicidal electromagnetic radiation.

### BACKGROUND OF THE INVENTION

In today's world, contamination by undesirable pathogenic microorganisms is a real and important danger. For example, news reports often present E. coli breakouts in such stock foodstuffs as Romaine lettuce. It has been reported that more than 50% of all grocery carts in grocery stores are contaminated with E. coli. Foodstuffs, such as vegetables and other produce may be contaminated by people who pick up and put down produce as they shop, thus potentially transferring undesirable microorganisms to the produce. It is also well known that hand contact is one of the most common methods of transferring contaminants. Wherever there is food there are possibilities of food contamination. Recently the Covid-19 pandemic has become a world wide crisis with rises in Covid-19 cases despite many draconian methods to limit the spread of the disease. Masks are currently one of the best methods to limit contamination, but even the best generally available masks are only 95% effective. Airborne cross-contamination occurs wherever people congregate, and even when people are practicing social distancing. Someone carrying the disease can pick up a box of cereal in a store, for example, contaminate it, put it back down and another person may pick it back up and place it in their shopping cart, thus possibly contaminating themselves and every item with which it comes into contact and is then brought into the home to cause exposure to the remainder of the family. Any container that carries objects that are dispersed to the general public is under suspicion for spreading such pathogens, including COVID-19.

Contamination is not limited to foodstuffs. Hospital-acquired germs, pathogens, viruses, infections and the like are also an issue in today's healthcare system. Hospitals are areas of congregation for people who are carrying pathogens and despite great efforts, hospitals continue to be contamination sources. Such pathogens and infections can lengthen hospital stays and increase health care costs. Hospital-acquired pathogen and infections have many causes, including the transmission pathogenic microorganisms by contaminated medical devices. For example, the American Medical Association has passed a resolution recommending that stethoscopes (and other hand-held medical instruments) be cleaned between uses. See American Medical Association House of Delegates. Proceedings of the 50 the Interim Meeting. Chicago, Ill.: American Medical Association; Dec. 8-11, 1996:398.

As well, other hospital devices, such as scalpels and surgical equipment, IV bags, attachments and poles, bedpans, and other devices and objects that are used in hospitals that have potential to attract and spread pathogenic microorganisms, all need cost-effective ways to get clean and disinfected.

There are a number of paths by which a pathogen, Covid-19 for example, can be transferred. For example, a moving van picking up, moving and unloading furniture to a home can cause a transfer even with the best of current controls. Other containers, such as truck trailers may also be avenues of disease transfer.

Thus, there is a real continuing need to improve our ability to stop or at least further limit the spread of pathogens and especially viruses from spreading, using every possible avenue, including devices such as described below.

### BRIEF DESCRIPTION OF THE FIGURES

FIG. 1 shows a graphic representation of a sanitizing bag of the current disclosure

FIG. 2 shows a graphic representation of a second embodiment of a sanitizing bag of the current disclosure.

FIG. 3 shows a graphic representation of a sanitizing insert of the current disclosure for a grocery cart

### BRIEF DESCRIPTION OF THE CURRENT DISCLOSURE

Disclosed herein are methods and devices that are designed to reduce or eliminate contamination by pathogens that occurs on objects, devices, medical equipment, foodstuffs, produce, clothing and the like.

In a first embodiment, disclosed and claimed herein are bags for sanitizing the contents of the bag comprising two to four walls comprised of material which prevents sanitizing radiation from passing through, at least one sanitizing radiation-emitting device attached to the interior sides of the walls and configured throughout the interior of the bag when more than one is present, wherein the sanitizing radiation is emitted into the interior of the bag when in operation and a sanitizing radiation reflective surface situated on the interior sides of the walls between the interior sides of the walls and the multiple sanitizing radiation-emitting devices, wherein the bag for sanitizing may optionally comprise a top and/or a bottom.

In a second embodiment, disclosed and claimed herein are bags of the above embodiment, wherein the sanitizing radiation devices on the side of the bag are evenly spaced.

In a third embodiment, disclosed and claimed herein are bags of the above embodiments further comprising a rechargeable battery removably attached to the bag configured with an electrical connection accessible from outside the bag for charging the batteries, wherein the battery operates the sanitizing radiation-emitting devices a well as any peripheral components which require electricity to operate.

In a fourth embodiment, disclosed and claimed herein are bags of the above embodiments further comprising an electrical connection that operates the sanitizing radiation-emitting devices a well as any peripheral components which require electricity to operate, using power from an external AC outlet.

In a fifth embodiment, disclosed and claimed herein are bags of the above embodiments further comprising sensors to sense any light that might enter the bag, a manual on/off switches to manually activate the sanitizing radiation emitting devices, automatic switches that activate the radiation emitting devices when the bag is closed, sensors have determine that no radiation will exit the interior of the bag, electronic control units for controlling the time of radiation emission or combinations thereof.

In a sixth embodiment, disclosed and claimed herein are bags of the above embodiments for use to sanitize foodstuffs, produce, containers that hold the foodstuffs and produce, the bag itself, medical equipment and devices, veterinarian medical equipment and devices, purses, handbags, pocketbooks, suitcases, knapsacks, camping equipment, and personal hygiene items.

In a seventh embodiment, disclosed and claimed herein are bags of the above embodiments, wherein the bag is configured to removably fit inside another bag or container.

In an eighth embodiment, disclosed and claimed herein are bags of the above embodiments wherein the bag is a liner comprising 4 walls, a bottom and a top cover configured to fit into a shopping cart wherein the radiation-emitting devices are arranged throughout the walls, bottom, top or a combination thereof, an optional reflective liner, and configured to emit radiation inside the bag.

In a ninth embodiment, disclosed and claimed herein are bags of the above embodiments, wherein the sanitizing radiation devices on the side of the bag are evenly spaced.

In a tenth embodiment, disclosed and claimed herein are bags of the above embodiments further comprising a rechargeable battery removably attached to the bag configured with an electrical connection accessible from outside the bag for charging the batteries, wherein the battery operates the sanitizing radiation-emitting devices a well as any peripheral components which require electricity to operate and further comprising an electrical connection that operates the sanitizing radiation-emitting devices as well as any peripheral components which require electricity to operate, using power from an external AC outlet and sensors to sense any light that might enter the bag, and further comprising manual on/off switches to manually activate the sanitizing radiation emitting devices, automatic switches that activate the radiation emitting devices when the bag is closed, sensors have determine that no radiation will exit the interior of the bag, electronic control units for controlling the time of radiation emission or combinations thereof.

In an eleventh embodiment, disclosed and claimed herein are bags of the above embodiments wherein the bag is configured to fit inside a grocery cart.

In a twelfth embodiment, disclosed and claimed herein are bags of the above embodiments wherein the bag is configured to fit inside the trailers of semis-tractor-trailer trucks, the holds of cargo ships, holds of airplanes, U-Haul® type conveyances, moving trucks, refrigerators, food lockers, freezers, the trucks of cars, restaurant food storage area, food displays in grocery stores, buses, train box cars, taxis, public conveyances, coolers, and hospital equipment storage areas.

### DETAILED DESCRIPTION OF THE CURRENT DISCLOSURE

Disclosed and claimed herein is a bag which has sanitizing radiation-emitting devices positioned throughout the interior sides of the walls of the bag positioned to emit sanitizing radiation into the interior of the bag so that when an object is placed inside the bag, sanitizing radiation may be impinged onto the object to allow for sanitization. The bag may be of any style, such as, for example, 2-sided, 3-sided or the conventional 4 -sided. The bag may include a bottom such as would be conventional for a 4-sided bag or not as would be conventional for a 2-sided bag, such 2-sided bag would be connected at the bottom and sides. The bag may also have a top configured to enclose the bag in such a manner that no sanitizing radiation escapes from the bag when the bag is closed and the sanitizing radiation-emitting devices are activated.

At least one sanitizing radiation-emitting device is attached to the interior wall of the sides of the bag. When more than one sanitizing radiation-emitting device is present, they are configured throughout the interior of the bag and situated to emit sanitizing radiation into the interior of the bag. The sanitizing radiation-emitting devices may be comprised of UV light emitting components. The UV light may be comprised of UVA, UVB and or UVC wavelengths or combinations thereof, hereafter called sterilizing radiation. Sanitizing radiation-emitting devices of the current disclosure may be LEDs, bulbs or other sterilizing radiation-emitting devices. The sanitizing radiation-emitting device may be one or more cold cathode sanitizing radiation-emitting lamps situated at ends of the bag and/or top of the bag, wherein the sanitizing radiation is emitted into the interior of the container. The walls of the bag of the current disclosure may also contain sanitizing radiation reflective surfaces situated on one or more of the interior sides of the walls between the interior sides of the walls and the at least one sanitizing radiation-emitting device. The sanitizing radiation-emitting devices may be evenly spaced on the inside of the bag walls or they maybe strategically spaced to allow higher levels of radiation in one area of the inside of the bag and less I other areas.

The bag further comprises a rechargeable battery which can be permanent or removable. The bag includes a electrical port to which the battery is attached as well as a connection for recharging the battery from a source outside the bag. The battery is configured to operate the sanitizing radiation-emitting devices as well and other electronic components of the bag,

It is common these days for consumers to provide their own grocery bags thus reducing the impact that grocery store-provided, single use bags has on the environment, such as, for example, plastic bags which take hundreds of years to decompose, or paper bags which put a stress on renewable resources such as trees. In one embodiment of the current disclosure, provided is a reusable grocery bag which is comprised of sterilizing devices comprised of UV light emitting components. The UV light may be comprised of UVA, UVB and or UVC wavelengths or combinations thereof, hereafter called sterilizing light or sterilizing radiation. It is well known in the art that these wavelengths kill or deactivate all or almost all microorganisms that are exposed to the wavelengths. The sanitizing radiation emitting components of the current disclosure may be LEDs, bulbs or other sterilizing radiation emitting components. In another embodiment of the current disclosure, provided is a reusable insertable bag which is comprised of sterilizing devices comprised of UV light emitting components and designed to be removably placed inside a bag or container.

In another embodiment of the current disclosure are bags that are configured to fit into grocery carts (most grocery carts have standardized dimensions) into which groceries, foodstuffs, packages and the like are placed, and which can then be sterilized.

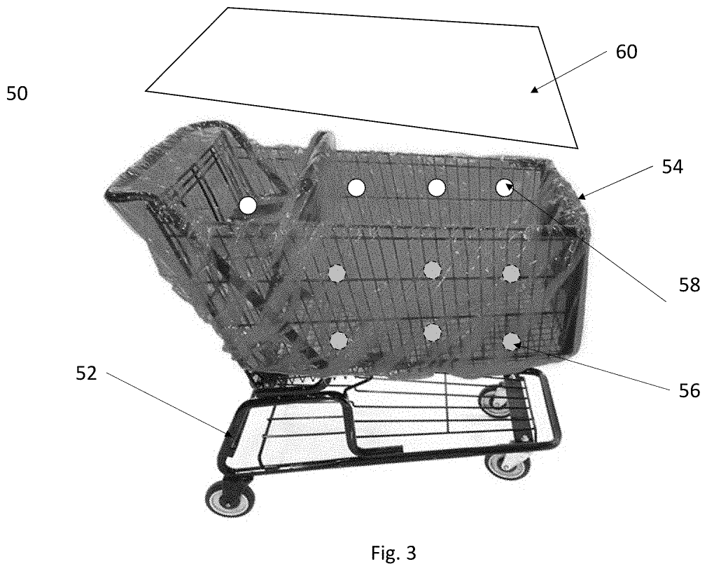

An example of one of the embodiments of the currently disclosed device is shown in FIG. 3 wherein a grocery cart 52 is fitted with a bag 54 of the current disclosure, the bag containing sanitizing radiation-emitting devices in a proximal position in the FIG. 56 and sanitizing radiation emitting devices in a distal position in the FIG. 58. A cover 60 is show, not attached.

The sanitizing bags, including grocery bags as well as other types of bags such as insertable bags, of the current disclosure, are configured to allow sterilizing devices to be placed anywhere on the surfaces of the inside of the sterilizing bag, any and all sides, bottom and top, if a top is part of the bag. Additionally, the sterilizing bags may be configured to allow insertion of the sanitizing bags inside other, outer bags or containers which may serve as sturdier bags for holding heavy object, the sanitizing bag being an “inner” bag. The sterilizing devices can be permanently attached to a sanitizing bag, such as a grocery bag, above and/or below the inner surfaces of the grocery bag. The inner surfaces of the sanitizing bag may be reflective of the sanitizing light/radiation. The reflecting surfaces may be smooth or that may be patterned, such as embossing, so that the reflecting light can be spreads out into areas that the sterilizing radiation can directly reach. The sanitizing bag or container not which it is placed is further configured to be closed and made light-tight so that no sterilizing radiation may escape. The sanitizing bag contains a power pack which is used to power the sanitizing radiation emitting components as well as wiring to interconnect the sanitizing radiation components, and other electronic components that may be present such as sensors, timers, alarm systems and the like. The bag may have a switch by which the user may manually turn the sterilizing radiation on. The grocery bag may further have a sensor that senses if any light from the outside enters the bag thus indicating that the bag is not fully lighttight, such a sensor is configured to not allow the sanitizing radiation emitting components to be turned on. The power pack comprise replaceable batteries or they may comprise rechargeable batteries and the accompanying mechanisms.

A further embodiment of the current disclose is a method whereby foodstuffs and/or produce or other items are placed in the sanitizing bag of the current disclosure, closing the bag and either manually or automatically having the sanitizing radiation turn on so that the sanitizing radiation impinges onto the contents of the sanitizing bag. The bag may contain electronic controlling unit, such as computing devices that determine the right amount of sanitizing radiation, determine sensor output and controlling the time and energy of sterilization as well as automatically turning off the sanitizing radiation when the controlling mechanism determines the set amount.

One area that is rarely addressed when using consumer supplied, reusable grocery bags is the issue of cleaning out the bag when all the contents are removed. If the bag is cleaned by hand the issue of thoroughness is always a concern. For example, a chicken is placed in the bag and although the sanitizing radiation is turned on during all or partial transport, there may be a few drippings of raw chicken fluid that drip back into the bag. If not thoroughly cleaned the residual material in the bag may contaminate any food that now with which it comes into contact. The current disclosure describes devices and methods to eliminate this issue. Another embodiment of the current disclosure is the sanitization of the sanitizing bag when not carrying foodstuffs or produce. The sanitizing radiation may be manually turned on and a timing device turns off the device automatically after a period of time chosen by the consumer, or the electronic controlling unit may automatically sanitize the interior of the bag when the empty bag is closed. The sanitizing bag may include lights and/or sounding devices to notify the user when the sanitization process is complete, as well as sensors to ensure the sanitizing radiation does not escape the bag.

The devices and methods of the current disclosure may also be applied to broader applications. For example, the interiors of trucks and truck trailers, airplane baggage and transport holds, railroad cars and other mass transportation systems that move foodstuffs and produce and other consumer products that come into contact with the general public and can be sterilized at any point in the transportation process. For example, a truck carrying foods and produce through warm conditions are prone to outbreaks of undesirable microorganism. Configuring the inside of a truck's trailer, walls, ceiling, floor with devices of the current disclosure will help to keep the foodstuffs and produce and other objects free of active undesirable microorganisms, such as, for example COVID-19. As well, when the truck's trailer or other transport container is empty the devices may serve to sanitize the interior of the truck trailer or other transport devices in order to prevent any possible cross-contamination that may occur during the off-loading of the cargo.

The same can hold true of any container that holds food products, such as storage bins in a restaurant or in a grocery store, as well as pantries, food displays at stores, refrigerators, freezers and the like.

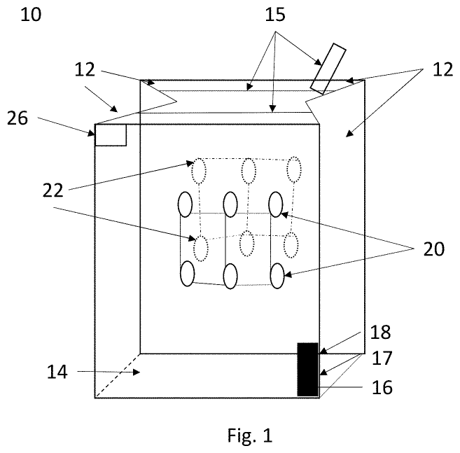

An example of one of the embodiments of the currently disclosed device is shown in FIG. 1. The reusable bag 10, has four sides 12 and a bottom 14. The bag contains a battery pack 16, including a USB charging port 17 as well as an external connector for powering from an AC or DC port 18. The batteries or power source operated the sanitizing radiation-emitting devices 20, devices in the proximal wall of the bag, and 22, devices in the distal wall of the bag, as well as lights, and other sensors, 26. In this embodiment a zipper 15 is configured to close the top of the bag so that no sanitizing radiation may escape.

In other embodiments, the current disclosure provides for devices for sanitizing medical equipment, such as, for example, doctor's bags, as well as nurse's bags. Nurse's bags, particularly those used by visiting nurses a well as front line personnel who are exposed routinely to viruses such as COVID-19, that treat patients and are required to carry away contaminated and potentially dangerous pathogens, can be partitioned to hold sterile materials in one petition and contaminated materials in the other petition. The current disclosure provides for sanitizing radiation devices configured throughout the interior of such containers as doctor's bags and/or nurse's bags with the radiation emitted directly into the interior and onto the equipment as well as contaminated materials.

In further embodiments, the current disclosure provides for sanitizing bags for use in hospitals and other facilities wherein medical treatment which requires sterile materials occurs, such as, for example clinics, EMT vehicles, rescue vehicle, ambulances, fire trucks, police cars, as well as public transportation, taxis, and the like, wherever the general public is present.

In hospitals, the current disclosure provides for bags for sanitizing hospital devices, such as scalpels and surgical equipment, IV bags, attachments and poles, bedpans, and other devices and objects that are used in hospitals that have potential to attract and spread pathogenic microorganisms. While there are processes and procedures for sterilization of medical equipment, the current disclosure provides for a second level of sterilization, as well to maintain the sterility of the previously sterilized equipment, such as, for example, during storage or after handling of the equipment to ensure that the equipment is as sterile at the time of use as is was when first being sterilized.

In other embodiments, the current disclosure provides for personal containers such as purses, pocketbooks, handbags and the like which are intended to hold cosmetics, hairbrushes, combs, make-up brushes and other items that are susceptible to contamination. In these embodiments, sanitizing radiation devices are configured throughout the interior of the purse, handbag, pocketbook and the like with the radiation emitted directly into the interior. Alternatively, disclosed herein are sanitizing bags which can be removably placed inside an existing personal container such as handbag, purse pocketbook, and the like.

In another embodiment of the current disclosure, provided are bags which are pouches, gym bags, duffle bags, suitcases and knapsacks, as well a sanitizing bags which can be removably placed inside such bags.

In other embodiments, the current disclosure provides for sanitizing bags for personal hygiene devices for use in sanitizing personal items such as, for example, toothbrushes, combs, glasses, goggles, make-up equipment, and the like.

In further embodiments, the current disclosure provides for sanitizing bags for use in veterinarian hospitals and other facilities wherein medical treatment of animals which requires sterile materials occurs, such as, clinics, EMT vehicles, rescue vehicle, ambulances, fire trucks, police cars, public transportation, taxis, buses, trains and the like.

In another embodiment of the current disclose is a method whereby foodstuffs, produce, medical equipment or other items that come not contact with the general public to be sanitized or items for maintaining sterilization are placed in the sterilizing bag of the current disclosure, closing the bag and either manually or automatically having the sanitizing radiation turn on so that the sanitizing radiation impinges onto the contents of the bag. The bag may contain electronic controlling unit, such as computing devices that determine the right amount of sanitizing radiation, determine sensor output and controlling the time and energy of sterilization as well as automatically turning off the sanitizing radiation when the controlling mechanism determines the set amount.

As used herein the term “bag” refers to generally soft, pliable, conformable containers made of flexible materials such as cloth, plastic or other polymeric materials but rigid enough to stand up by itself or when placed inside another bag, container or other device into which the sanitizing bag is placed. The term includes containers in which less than all sides of the container are soft, for example, a bottom and/or less than all sides are made of a hard, structural material. The term “bag” is not limited to small containers, but is extends to include small and large configurations, such as a bag large enough to fit inside a trailer of a semi-tractor-trailer-truck and other large container, such as the holds of cargo ships.

As used herein the term “multiple” means more than one.

The sanitizing bags of the current invention may include a rechargeable battery removably attached to the bag, including an electrical connection accessible from outside the bag for charging the batteries. The electrical connection may be a USB connection for recharging or may be a connection for using power from an external AC outlet.

The sanitizing bags of the current disclosure are configured to be closed to make them “light-tight” so that no sanitizing radiation may escape when in sanitizing mode. This includes light-tight zippers, flaps to enclose the opening through which items are placed in the bag, or other methods well known in the art to prevent light from escaping a container.

The interior of the sanitizing bags may be made of material configured to reflect UV sanitizing radiation, such as, for example, aluminum, either smooth or embossed in which the radiation ore effectively is scattered around the inside on the bag.

The sanitizing radiation emitting devices may be removably attached to the bag sides. They may be inserted into the walls of the bag as permanent fixtures or removable. The radiation emitting devices may be attached to an insert, such as a lining, which is then fitted inside the container or outer bag and can be readily removable.

The sanitizing bags of the current disclosure may have sensors to sense any light that might enter the bag as an indicator that the bag is not light-tight.

The bags of the current disclosure may have manual on/off switches to manually activate the sanitizing radiation-emitting devices, automatic switches that activate the radiation emitting devices when the bag is closed, sensors have determine that no radiation will exit the interior of the bag, electronic control units or combinations thereof.

The electronic control units may be comprised of logic devices which can monitor the radiation statistics, including, for example, time of exposure as well as the ability to wirelessly connect to control devices outside the bag.

The bag may further contain an electrical connection that operates directly from an outside AC or DC source, bypassing any battery component. The bag may also combine both a battery and a direct Ac or DC source.

The bag may contain sensors that aid in the operation of the bag such as, for example, lights sensors to determine if any light is entering the bag which could indicate that the bag s not fully closed thus allowing sanitizing radiation from exiting the bag when in operation. Other components of the bag may include automatic switches that turn the radiation devices on when the bag is closed, and it is determined that the bag is safe from radiation leakage. Electronic control units may also be present to provide timing, power sensing and the like.

In a further embodiment the sanitizing bags of the current disclosure as described about may have one or more cold cathode sanitizing radiation emitting lamps situated in the bags, such as, for example, at the ends of the top of the bag, wherein the sanitizing radiation is emitted into the interior of the container. These lamps may be used alone or in conjunction with the sanitizing radiation emitting LEDs as described above.

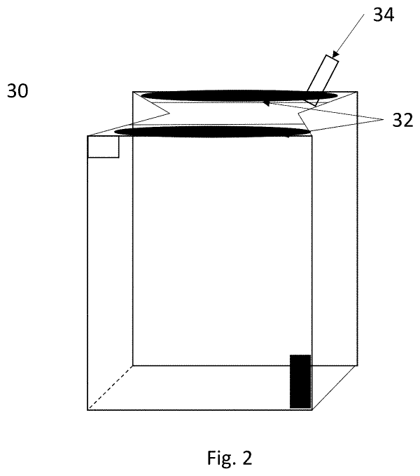

An example of an embodiments of the currently disclosed device using cold cathode lamps is shown in FIG. 2. The bag 30 is similar to the bag 10 in FIG. 1 except FIG. 2 has two cold cathode sanitizing radiation emitting lamps 32 situated next to, and beside the zipper 34 opening in such a manner as to allow the bag to be zippered closed and prevent sanitizing radiation from escaping, with the sanitizing radiation-emitting devices removed.

In another embodiment of the current disclosure, provided is a reusable insertable bag which is comprised of sterilizing devices comprised of UV light emitting components and designed to be removably placed inside a bag or container.

In another embodiment the bag of the current disclosure maybe extended to include large containers, such as, for example, the trailers of semis-tractor-trailer trucks, the holds of cargo ships, holds of airplanes, U-Haul® type conveyances, moving trucks, refrigerators, food lockers, freezers, the trucks of cars, restaurant food storage area, food displays in grocery stores, buses, train box cars, taxis, public conveyances, coolers, hospital equipment storage areas, and the like.

The sanitizing bag of the current disclosure may be a stand-alone bag or it may be configured to be an insert into other bags or containers allowing the conversion of any bag or container to a sanitizing bag or container. In some aspects of the disclosure the bag may nominally “fit” inside the bag or container with clips or other attachments configured on the sanitizing bag to obtain a suitable fit. The sanitizing bag may also be configured to fit almost exactly to the bag or container into which it is placed.

We claim:

1. A bag for sanitizing the contents of the bag comprising,
a. two to four walls comprised of material which prevents sanitizing radiation from passing through,
b. at least one sanitizing radiation-emitting device attached to the interior sides of the walls and configured throughout the interior of the bag when more than one is present, wherein the sanitizing radiation is emitted into the interior of the bag when in operation, and
c. a sanitizing radiation reflective surface situated on the interior sides of the walls between the interior sides of the walls and the multiple sanitizing radiation-emitting devices,
wherein the bag for sanitizing may optionally comprise a top and/or a bottom.

2. The bag of claim 1, wherein the sanitizing radiation devices on the side of the bag are evenly spaced.

3. The bag of claim 1 further comprising a rechargeable battery removably attached to the bag configured with an electrical connection accessible from outside the bag for charging the batteries, wherein the battery operates the sanitizing radiation-emitting devices a well as any peripheral components which require electricity to operate.

4. The bag of claim 1 further comprising an electrical connection that operates the sanitizing radiation-emitting devices a well as any peripheral components which require electricity to operate, using power from an external AC outlet.

5. The bag of claim 1, further comprising sensors to sense any light that might enter the bag.

6. The bag of claim 1, further comprising manual on/off switches to manually activate the sanitizing radiation emitting devices, automatic switches that activate the radiation emitting devices when the bag is closed, sensors have determine that no radiation will exit the interior of the bag, electronic control units for controlling the time of radiation emission or combinations thereof.

7. The bag of claim 1 for use to sanitize foodstuffs, produce, containers that hold the foodstuffs and produce, the bag itself, medical equipment and devices, veterinarian medical equipment and devices, purses, handbags, pocketbooks, suitcases, knapsacks, camping equipment, and personal hygiene items.

8. The bag of claim 1, wherein the bag is configured to removably fit inside another bag or container.

9. The bag of claim 8 wherein the bag is a liner comprising 4 walls, a bottom and a top cover configured to fit into a shopping cart wherein the radiation-emitting devices are arranged throughout the walls, bottom, top or a combination thereof, an optional reflective liner, and configured to emit radiation inside the bag.

10. The bag of claim 8, wherein the sanitizing radiation devices on the side of the bag are evenly spaced.

11. The bag of claim 8, further comprising a rechargeable battery removably attached to the bag configured with an electrical connection accessible from outside the bag for charging the batteries, wherein the battery operates the sanitizing radiation-emitting devices a well as any peripheral components which require electricity to operate.

12. The bag of claim 8, further comprising an electrical connection that operates the sanitizing radiation-emitting devices as well as any peripheral components which require electricity to operate, using power from an external AC outlet.

13. The bag of claim 8, further comprising sensors to sense any light that might enter the bag.

14. The bag of claim 8, further comprising manual on/off switches to manually activate the sanitizing radiation emitting devices, automatic switches that activate the radiation emitting devices when the bag is closed, sensors have determine that no radiation will exit the interior of the bag, electronic control units for controlling the time of radiation emission or combinations thereof.

15. The bag of claim 8 configured to fit inside a grocery cart.

16. The bag of claim 8 configured to fit inside the trailers of semis-tractor-trailer trucks, the holds of cargo ships, holds of airplanes, U-Haul® type conveyances, moving trucks, refrigerators, food lockers, freezers, the trucks of cars, restaurant food storage area, food displays in grocery stores, buses, train box cars, taxis, public conveyances, coolers, and hospital equipment storage areas
